(19) United States
(12) Patent Application Publication
(10) Pub. No.: US 2021/0346460 A1
(43) Pub. Date: Nov. 11, 2021

(54) TREATMENTS FOR COVID-19-RELATED SYSTEMIC SCLEROSIS, VASCULAR INSUFFICIENCY, DISTAL ISCHEMIA, AND RELATED DISEASE CONDITIONS

(71) Applicant: John L. COUVARAS, Paradise Valley, AZ (US)

(72) Inventor: John L. COUVARAS, Paradise Valley, AZ (US)

(21) Appl. No.: 17/313,977

(22) Filed: May 6, 2021

Related U.S. Application Data
(60) Provisional application No. 63/021,338, filed on May 7, 2020.

(51) Int. Cl. A61K 38/08; A61K 31/198; A61K 45/06

(52) U.S. Cl. CPC A61K 38/085; A61K 45/06; A61K 31/198

(57) ABSTRACT

A medical composition for treatment of a peripheral vascular disease is provided. The medical composition may include a dosage of a GABA-a agonist, a dosage of an Angiotensin II modulator, and a dosage of a Direct Oral Anticoagulant. The Angiotensin II modulator may include at least one of an ARB and an ACE Inhibitor, the ARB being an Angiotensin II, type 1 receptor antagonist. A method for treating a patient with a microvascular disease condition is provided. The patient may be provided, the medical composition may be provided, and the medical composition may be administered. The step of providing the patient may include providing a patient with at least one of systemic sclerosis, vascular insufficiency, distal ischemia, COVID-19 infection, and sepsis.

This Application claims the benefit of U.S. Provisional Patent Application No. 63/021,338, filed May 7, 2020, the disclosure of which is hereby incorporated by reference in its entirety.

### TECHNICAL FIELD

This application relates to compositions methods and systems relating to the release and/or production of prostacyclin and/or other vasodilators. More specifically, this application relates to compositions and related methods intended to reduce blood pressure in individuals suffering from essential hypertension; to treat and/or prevent bowel disorders; to treat and/or prevent peripheral vascular disease; and/or otherwise improve human health as described herein. The teachings of this disclosure may also be also applicable to reduce blood pressure, improve blood flow, and/or otherwise improve the heath of non-humans.

### BACKGROUND

Hypertension in humans is a condition wherein the blood pressure is elevated. Essential hypertension (“eHTN”) is hypertension without a known secondary cause (e.g., kidney disease). eHTN may result from various causes, including, for example, the constriction of the blood vessels. According to the CDC, approximately 33.2% of adults in the United States are being treated for hypertension. Essential hypertension is associated with 32.8 million physician office visits and 1.3 million emergency department visits per year. Additionally, there are approximately 35,000 deaths per year from eHTN (and hypertensive renal disease). Although various eHTN treatments have been used for decades, and billions of dollars' worth eHTN-treating pharmaceuticals are sold annually in the US alone, there is substantial room for improvement. Pharmaceutical companies continue to expend substantial resources toward improved eHTN treatments. Accordingly, there remains a need for additional eHTN treatments that are more effective, result in less side effects, and/or are cheaper.

It is estimated that 10-15% of Americans suffer from Irritable Bowel Syndrome (“IBS”); and that 1-2% of Americans suffer from Irritable Bowel Disease (“IBD”), such as Crohn's disease or ulcerative colitis. While there are a wide range of treatments available for these and other related bowel conditions, such treatments are typically invasive, involve a severe drug regimen, and/or offer insufficient relief for patients. Accordingly, there remains a need for additional IBS, IBD, and other bowel condition treatments that are more effective, result in less side effects, and/or are cheaper than the current standards of care.

There is also a need for additional, improved treatment options for peripheral microvascular disease; microangiopathic disease; systemic sclerosis; raynaud's digital ulcerations; distal necrosis, for example, resulting from microvascular compromise in chronic and acute conditions such as infection (e.g., from COVID-19 infections or other causes); and/or related maladies.

Diabetes is a metabolic disorder that is associated with microangiopathic disease. While ongoing blood glucose testing and corresponding provision of insulin and/or glucose may keep diabetes in check for many patients, this common treatment regimen has room for improvement.

Angiotensin II (“AII”), is a potent vasoconstrictor in the human body. It is part of the Renin-Angiotensin system and is activated to increase blood pressure via constriction of the vessels. Its release may be triggered by, for example, hypovolemia, renal hypoperfusion, and pregnancy. Low volume and dropping blood pressure may cause Angiotensin II to be induced in significant amounts. Angiotensin II is formed from Angiotensin I in the blood by an Angiotensin Converting Enzyme (“ACE”), which increases the production of AII. Conversely, a reduction in ACE may correspondingly reduce AII, and thereby lessen blood vessel construction and blood pressure. Angiotensin Converting Enzyme 2 (“ACE2”) is an enzyme that supports degradation of AII, and results in the formation of angiotensin (ANG 1-7), which opposes the actions of AII.

Peripheral and central blood vessels contain Angiotensin II receptor, type 1 (“AT1”) receptors and Angiotensin II receptor, type 2 (“AT2”) receptors.

AT1 receptors mediate certain cardiovascular effects of angiotensin II, notably vasoconstriction, which is an important mechanism for controlling blood pressure and volume in the cardiovascular system. That is, AII binding to AT1 receptor is considered to be associated with constriction. The binding of the AT1 receptor with AII is associated with variable vasoconstriction in normotensive and hypertensive humans, as well as in pregnant humans. Individuals in all of these categories may suffer from a variety of disease states related to the vasculature. Blood vessels are also known to make thromboxane and prostaglandins; adding aspirin-like compounds may affect the ratio of clotting constrictors to vessel dilators in unpredictable outcomes.

By contrast, AII binding to AT2 is associated with relaxation of blood vessels, which may also lower blood pressure.

ARBs are Angiotensin II, type 1 receptor antagonists. That is, they block the activation of AT1 receptors. Blockage of AT1 receptors prevents vasoconstriction, reduces secretion of vasopressin, and reduces production and secretion of aldosterone, amongst other actions. The combined effect is generally understood to reduce constriction of blood vessels. AT1 receptor blockers (ARBs) are associated with reduction in blood pressure in HTN humans and are believed to reduce constriction (as opposed to increasing vasodilation).

Angiotensin-converting enzyme inhibitors (ACE-I) block the angiotensin converting enzyme (ACE), which otherwise facilitates the formation of AII from Angiotensin 1. Accordingly, ACE-I may reduce the level of circulating AII, and thus reduce the activation of AT1 (and AT2) receptors by AII. In turn, the resulting reduction of AII binding to AT1 receptors is believed to reduce vessel contractility (as opposed to increasing vasodilation).

Thus, use of ACE-I and/or ARBs may facilitate reduced contraction of blood vessels, indirectly supporting vasodilation and making it easier for blood to flow through the vessels, which, in turn, may reduce blood pressure. The literature provides that this result is due to reduction in smooth muscle constriction.

In eHTN patients, high blood pressure often naturally reduces AII to negligible levels in circulation. This suppression in AII is due to feedback on the Renin-Angiotensin System. However, adding an ARB to block the remaining very low levels of AII from being able to bind to the AT1 receptor appears to be associated with lowered blood pressure. This is common knowledge in medicine, and the justification for giving ARBs to eHTN patients. Paradoxically, ACE-Is have generally been shown to lower blood pressure in eHTN patients even though eHTN is already associated with low levels of AII.

Both ARBs and ACE-Is have side effects which are not desirable. For example, ARBs are associated headaches, fainting, and dizziness; ACE-Is are associated with skin rashes and a persistent dry cough. Ultimately, it would be better if these substances were used smaller doses. However, reducing dosages tends to also reduce their effectiveness.

Endothelium Relaxing Factor (“EDRF”) is the major endothelium-derived relaxing factor in small-resistance rat mesenteric arteries and human gastroepiploic arteries that are known to control vascular tone and blood pressure. EDRF is generally understood to be nitric oxide (“NO”). In both the pregnancy and hypertensive literature, nitric oxide release has been shown to respond, in both uterine and systemic vessels, to various stimuli such as shear stress, estrogen, progesterone, and angiotensin II binding to the AT2 receptor on blood vessels.

Prostaglandin I₂ (“PGI2”), also referred to as prostacyclin, is vasodilator compound known to relax smooth muscle, and accordingly is associated with blood pressure reduction. Compared with EDRF, PGI2 is understood to contribute very little to endothelium-dependent relaxation of rat aorta and large proximal branches of mesenteric arteries and human proximal gastroepiploic arteries. In addition, in vivo data have shown that endogenous PGI2 may have little role in the regulation of blood pressure compared with EDRF because pharmacological blockade of Prostaglandin-Endoperoxide Synthase, also known as Cyclooxygenase (“COX”), with aspirin or indomethacin does not substantially affect resting blood pressure in humans or rats. By contrast, pharmacological blockade or genetic ablation of NO synthase has a profound effect on blood pressure, increasing it by 30 to 50 mm Hg. The prior art does not indicate or suggest that AII levels affect PGI2 secretions in pregnant systemic vessels.

GABA has been reported to lower blood pressure in eHTN humans. However, the exact mechanisms underlying GABA action are still largely unknown. For example, GABA has no effect to reduce blood pressure in normotensive patients.

There are two classes of GABA receptors, GABA-a receptors and GABA-b receptors. GABA-a receptors are found in peripheral vessels of normotensive, hypertensive, and pregnant humans. Both receptors have been demonstrated peripherally, but GABA-a has been limited to autonomic ganglia, pancreatic tissue, adrenal medulla, renal vessels, uterine myometrium, cerebral vessels, mesenteric vessels, and pulmonary artery smooth muscle.

Classically, the GABA-a receptor is noted in both CNS (central nervous system) and periphery. It modulated chloride ion channels and is a hetero-tetramer of two alpha and two beta proteins which form a ligand. There are now various protein chains of alpha, beta, delta and gamma and a myriad of combinations. There appear to be binding sites for progesterone or metabolites, and these modify GABA affinity and action. A GABA-a agonist may be defined as a compound that will bind to a GABA-a receptor and exert a positive action. GABA may be considered a GABA-a agonist.

The GABA-b receptor was discovered later by its higher binding affinity. It is calcium dependent and G-protein mediating. Because of inherent difficulties in displaying GABA-b binding in the presence of GABA-a receptors, the GABA-b receptor was only later noted in the peripheral tissues.

GABA-a agonists have been reported to relax pulmonary and mesenteric smooth muscle. However, the mechanism has only been identified with NO release, not that of PGI2. Another study concluded that that the vaso-relaxatory effect of GABA in diabetic vessels is mediated by the GABA-b receptor and nitric oxide. Other scientific literature dating back to the 1950s indicated that GABA injected intravenously produced a drop in blood pressure and bradycardia, although there were species response differences.

GABA injection, depending upon the amount, is understood to have a dual character of depressor or pressor on blood pressure: Lower doses tend to increase blood pressure, while higher doses tend to reduce it. Furthermore, differences between anesthetized and unanesthetized animals regarding GABA's depressant action have been demonstrated. Oral GABA administration does not cause any depressant effect on blood pressure of normotensive animals, but in hypertensive subjects it has been shown to lower blood pressure significantly and maintain it as long as oral supplementation was continued.

It has been proposed that that GABA's cardiovascular effects may have been due to peripheral ganglionic blocking action, but later results were not consistent with such a hypothesis. Other studies to determine the local effect of GABA upon vessels suggested that the cerebral circulation did respond via GABA-a, a low affinity receptor, to cause relaxation. This has been seen in intact feline vessel segments. No vasomotor action by GABA has been shown in various vessels, such as the aorta, pulmonary artery, mesenteric artery, and internal carotid artery.

### SUMMARY

The present disclosure provides a description of compounds, methods, and treatments to address the perceived problems described above. More specifically, disclosed embodiments may promote the release of prostacyclin or other vasodilators into the blood stream to increase vasodilation, and thereby improve blood flow, lower blood pressure, and/or improve a variety of medical conditions as a result. Various embodiments of the disclosure may cause or support increasing PGI2 levels and advantageous corresponding dilation effects in various systemic vessels, including mesenteric/splanchnic vessels and the peripheral systemic microvasculature.

In one embodiment, a medical composition for treatment of a peripheral vascular disease is provided. The medical composition may include a dosage of a GABA-a agonist, a dosage of an Angiotensin II modulator, and a dosage of a Direct Oral Anticoagulant. The Angiotensin II modulator may include at least one of an ARB and an ACE Inhibitor, the ARB being an Angiotensin II, type 1 receptor antagonist.

The medical composition may include a dosage of at least one NO synthetase promoter. The at least one NO synthetase promoter may include at least one of biopterin (BH4), arginine, and citrulline.

The medical composition may include a dosage of at least one arginase inhibitor. The at least one arginase inhibitor may include at least one of Ornithine and Valine.

The medical composition may include a dosage of at least one of a NO synthetase promoter and an arginase inhibitor and/or a dosage of at least one AT2 agonist. The at least one AT2 agonist may include at least one of C21 peptides, Saralasin, and Sarile. The at least one of Saralasin and Sarile may serve as both an AT2 agonist and an ARB.

The dosage of the Direct Oral Anticoagulant within the composition may be of an amount that is sub-therapeutic with respect to other medically accepted modes of Direct Oral Anticoagulant use.

The medical composition may further include a dosage of at least one EO-regulating compound.

In another embodiment, a method for treating a patient with a microvascular disease condition is provided. The method may include steps of providing the patient, providing a medical composition, and administering the medical composition to the patient. The medical composition may include a dosage of a GABA-a agonist, a dosage of an Angiotensin II modulator, and a dosage of a Direct Oral Anticoagulant. The Angiotensin II modulator may include at least one of an ARB and an ACE Inhibitor, the ARB being an Angiotensin II, type 1 receptor antagonist.

The method may further include reducing at least one of bradykinin levels and clotting agents through action of the dosage of the Direct Oral Anticoagulant and increasing prostacyclin release in blood vessels affected by the vascular disease condition through combined action of the dosages of the GABA-a agonist and Angiotensin II modulator.

The medical composition may further include a dosage of at least one NO synthetase promoter; the method may further include a step of increasing EDRF release through action of the dosage of the at least one NO synthetase promoter.

The medical composition may further include a dosage of at least one arginase inhibitor; the method may further include a step of increasing EDRF release through action of the at least one arginase inhibitor.

The medical composition may further include a dosage of at least one AT2 agonist; the method may further include a step of increasing EDRF release through action of the at least one AT2 agonist.

The step of providing the patient may include providing a patient with a microvascular disease condition of at least one of systemic sclerosis, vascular insufficiency, and distal ischemia; providing a patient infected with COVID-19; and/or providing a patient with sepsis. For example, the step may include confirming that the patient has at least one such condition.

### BRIEF DESCRIPTION OF THE DRAWINGS

The accompanying drawings, which are incorporated into and constitute a part of this specification, illustrate several embodiments and aspects of the compounds, methods, and treatments described herein and, together with the description, serve to explain the principles of the invention.

FIG. 1A is a graph of laboratory data indicating PGI2 response in pregnant uterine arteries at various levels of Ouabain, wherein GABA is provided and Taurine is not—both without and with the provision of AII.

FIG. 1B is a graph of laboratory data indicating PGI2 response in pregnant uterine arteries at various levels of Ouabain, wherein both GABA and Taurine are provided—without and with the provision of AII.

FIG. 2A is a graph of laboratory data indicating PGI2 response in intact nonpregnant systemic vessels at various levels of GABA, wherein neither Taurine nor Ouabain is provided—without and with the provision of AII.

FIG. 2B is a graph of laboratory data indicating PGI2 response in intact nonpregnant systemic vessels at various levels of GABA, wherein neither Taurine nor AII is provided—without and with the provision of Ouabain.

FIG. 2C is a graph of laboratory data indicating PGI2 response in intact nonpregnant systemic vessels at various levels of GABA, wherein AII is provided and Taurine is not—without and with the provision of Ouabain.

FIG. 3A is a graph of laboratory data indicating PGI2 response in pregnant uterine arteries at various levels of GABA, wherein neither Taurine nor Ouabain is provided—without and with the provision of AII.

FIG. 3B is a graph of laboratory data indicating PGI2 response in pregnant systemic arteries at various levels of GABA, wherein neither Taurine nor Ouabain is provided—without and with the provision of AII.

FIGS. 4A and 4B are graphs of laboratory data illustrating PGI2 response to taurine levels, GABA levels, and the provision of AII in pregnant uterine vessels with lower and higher Ouabain conditions, respectively.

FIGS. 5A is a graph of laboratory data illustrating PGI2 response in pregnant systemic arteries to the provision of various combinations of GABA, AII, and bicuculine.

FIGS. 5B is a graph of laboratory data illustrating PGI2 response in pregnant uterine arteries to the provision of various combinations of GABA, AII, and bicuculine.

FIG. 6 is a graph illustrating changes in PGI2 response versus basal levels, in both pregnant uterine vessels and pregnant systemic vessels, when the endothelium is removed.

FIG. 7 is a representative illustration of an inverted PGI2 release effect in different vascular circuits compartments during pregnancy.

FIGS. 8A and 8B are graphs and descriptions of laboratory data indicative of GABA distribution in various pregnant uterine tissues and compartments.

### DETAILED DESCRIPTION

In various embodiments, compounds, compositions, and treatment methodologies disclosed herein may be utilized to treat or prevent eHTN.

In other embodiments, they may be utilized to improve blood flow to mesenteric/splanchnic circuit. In turn, treatment and/or prevention of reduced blood flow in the mesenteric/splanchnic circuit may effectively treat, prevent, and/or otherwise address IBS, IBD, and related bowel conditions.

In yet other embodiments, they may be utilized to improve blood flow in other peripheral system circuits, for example, the peripheral arteriolar circuit. In turn, treatment and/or prevention of reduced blood flow may effectively treat, prevent, and/or otherwise address peripheral microvascular disease; microangiopathic disease; systemic sclerosis; raynaud's digital ulcerations; distal necrosis—for example, resulting from microvascular compromise in chronic and acute conditions such as infection (e.g., from COVID-19 infections or other causes); and/or related maladies. Specifically, the compounds and methods disclosed may advantageously result in improved blood flow in the peripheral systemic circuit affected by one or more specific diseases discussed herein.

In yet other embodiments, compounds, compositions, and treatment methodologies disclosed herein may be utilized to improve blood flow and/or prevent clotting in peripheral system circuits of patients suffering from adult onset diabetes, suffering from other metabolic syndromes, and/or at risk of clotting.

### Co-Administered Compounds and Composition Thereof

In certain embodiments, a patient may be provided with a dosage of GABA-a agonist(s) and a dosage of an Angiotensin II modulator (e.g., an ARB or ACE-I) to promote PGI2 production and/or release. Such provision may, in some embodiments may be made by way of a single composition, for example in a tablet, but this disclosure is not so limited. GABA, Abecarnil, Eszopiclone, Bamaluzole, Fengabine, Gabamide, GABOB, Gaboxadol, Ibotenic acid, Isoguvacine, Isonipecotic acid, Muscimol, Pantherine, Phenibut, Picamilon, Progabide, Propofol, Quisqualamine, SL 75102, Thiomuscimol, Topiramate, and Zolpidem are examples of GABA-a agonists and may be suitable.

ARBs and ACE-Is tend to have a relatively long half-life in an individual's system. By contrast, a GABA-a agonist(s) has a relatively short half-life. Thus, in some preferred embodiments, the composition may be provided in a time release form, with the GABA-a agonist(s) being released over a longer period of time. For example, a tablet can be provided which includes a dosage of ARB and/or ACE-I in an outer layer, and inner layer(s) of GABA-a agonist(s), which release over a period of time. In this manner, the ARB and/or ACE-I may be delivered into the system and remain there for a period of time. The GABA-a agonist(s), having a shorter time in an individual's system, may be released over that period of time to provide a constant source of GABA-a agonist(s) to complement the ARBs and/or ACE-Is in activating the biological mechanisms discussed herein. It will be understood by one skilled in the art that various delivery mechanisms for releasing GABA-a agonist(s) over a period of time that ARBs and/or ACE-I are present in the system can be employed, such as capsules, tablets, IV administration, and the like.

In certain embodiments, one or more endogenous ouabain (“EO”)-regulating compound(s) may be further included in the composition or otherwise provided to a patient. As discussed below, this may further promote PGI2 production and/or release. For example, certain low dose steroids may suppress adrenal endogenous ouabain. And, digitoxin, digoxin, and/or similar compounds may compete with endogenous ouabain, and thereby lower EO activity and enable higher PGI2 formation and release. Glucocorticoids, with or without salt modulating activities, are examples of low dose steroids that may be expected to suppress adrenal endogenous ouabain and may be suitable. In various embodiments, digoxin, digitoxin, β-acetyldigoxin and methyldigoxin, another low dose glucocorticoid, and/or other Glycoside antagonists, may be utilized as EO-regulating compounds. Because digoxin and most EO-regulating compounds have long half-lives, they need not, in many embodiments, be included in a time-release form. However, certain EO-regulating compounds, for example, Nanomolar Ouabain, act quickly, and may advantageously be provided in a time-release form.

In some embodiments, arginase inhibitors and/or NO synthetase promoters may be further included in the composition or otherwise provided to a patient. As discussed below, the addition of arginase inhibitors and/or NO synthetase promoters may serve to redirect arginine metabolism away from spermine/spermidine metabolism and/or toward NOS to NO metabolism, and thereby increase EDRF release. In this manner, blood flow may be substantially improved via a synergistic combination of increased EDRF and PGI2 release. Biopterin (BH4), arginine, and citrulline are examples of NO synthetase promoters and may be suitable. Ornithine and Valine are examples of arginase inhibitors and may be suitable. In certain embodiments, providing one or more NO synthetase promoter(s) and/or arginase inhibitor(s) in a time-release form may be preferred, but is not believed to be necessary.

In some embodiments, Direct Oral Anticoagulants (“DOACs”) may be further included in the composition or otherwise provided to a patient. The addition of DOACs may serve to reduce clotting, lower bradykinin, and/or increase EDRF. In this manner, blood flow may be substantially improved via a combination of EDRF and PGI2 release, as well as clotting reduction and/or bradykinin-related smooth muscle contractions. Dabigatrab (Pradaxa), Rivaroxaan (Xarelto), Apixaban (Eliquis), and Edoxaban (Savaysa) are non-limiting examples of DOACs that may be suitable. Administration of the DOAC in a pill form may be required twice a day; additionally or alternatively, the DOAC may be provided in a time-release form.

In some embodiments, an AT2 agonist(s) may be further included in the composition or otherwise provided to a patient. The provision of one or more AT2 agonists may promote EDRF production and/or release. For example, C21 small peptides, may be included in composition embodiments to promote the production and/or release of NO. Saralasin and Sarile are additional examples of AT2 agonists and may be suitable: As these substances also function as ARBs, they may be characterized as ARBs and/or AT2 agonists. In certain preferred embodiments, AT2 agonists may be provided in a time-released form.

In some embodiments, an ACE-I may be utilized instead of or in addition to an ARB. However, as discussed below, blocking AII production with an ACE-I may limit the vasodilatory pathway with AT2 binding to make NO. Accordingly, it may be preferred to utilize an ARB to the exclusion of an ACE-I in certain circumstances and in certain compositions.

Compounds and/or compositions thereof described herein may be administered in various dosages, such as GABA-a agonist doses of 0.01 mg /day to 10 mgs/day; ARB (and/or ACE-I) doses of 0.01 mg/day to 1000 mgs/day; arginase inhibitor dosages of 0.01 mg/day to 1000 mgs/day; NO synthetase promoter dosages of 0.01 mg/day to 1500 mgs/day; DOAC dosages of 0.01 mg/day to 1000 mgs/day; and/or AT2 agonist dosages of 0.01 mg/day to 1000 mgs/day. It is contemplated that L-arginine may be provided in a dosage from 1 mg/day to 500 mg/day, and that biopterin (BH4) may be provided in a dosage from 1 mg/day to 1500 mg/day.

In narrower embodiments, compounds may be provided in amounts below or far below their typically effective dosages, but would still be clinically effective with respect to the biological mechanisms discussed herein in combination with other relevant compounds. For example, a typical daily ARB dosage is between 10 mg-100 mg; in various embodiments, an ARB dosage (provided in combination with a GABA-a agonist) may be between 0.01 mg/day to 10 mgs/day, more narrowly between 0.01 mg/day to 8 mgs/day, 0.01 mg/day to 5 mgs/day, or even 0.01 mg/day to 3 mgs/day. In another example, a typical daily DOAC dosage is between 25 mg-75 mg provided twice daily; in various embodiments, an DOAC dosage (provided in combination with a GABA-a agonist and an ARB (or ACE-I)) may be between 0.01 mg/day to 50 mgs/day, more narrowly between 0.01 mg/day to 40 mgs/day, 0.01 mg/day to 20 mgs/day, or even 0.01 mg/day to 10 mgs/day.

Ultimately, it is contemplated that appropriate dosages of each compound may be dependent on a patient's weight, particular medical condition, and/or the particular grouping of compounds administered. Consistent with this disclosure, a person of ordinary skill in the art could determine appropriate effective dosages of each compound based on these characteristics.

The aforementioned compounds and compositions thereof may be administered orally and/or intravenously. It is contemplated that they may be dosed once, twice, or thrice daily.

### Methods of Use

In certain embodiments, methods of treatment may include a step of identifying a patient with (and/or a substantial risk for) a certain disease or condition related to blood flow or blood pressure issues; a step of co-administering doses of appropriate compounds to the patient; a step of increasing the release of vasodilatory compounds in systemic vessels; a step of increasing vasodilating of relevant vessels by relaxation of smooth muscle to lower blood pressure and/or improve blood flow; and a step of treating (or preventing) the disease or condition as a result.

The step of identifying may include, for example, identifying a patient with eHTN, a bowel disease or condition, a microvascular disease or condition, a metabolic disease or condition, COVID-19, and/or another malady substantially related to high blood pressure and/or blood flow problems—and/or identifying a patient with a substantial risk for developing one of the aforementioned conditions.

In some embodiments, for example where co-administration of EO-regulating compounds is contemplated additional steps of testing a patient's EO level, and comparing the test results to threshold EO values may be included in methods of treatment.

The step of co-administering dosages appropriate compounds may be accomplished by administering a composition of the appropriate compounds. In preferred embodiments, the co-administered dosages may include at least a dosage of ARB (and/or ACE-I) and a dosage of a GABA-a agonist. Dosages of one or more EO-regulating compounds, DOACs, arginase inhibitors, NO synthetase promoter(s), and/or AT2 agonist(s) may also be co-administered, as appropriate.

The step of increasing the release of vasodilatory compounds in systemic vessels may be characterized by increased release of PGI2 in preferred embodiments. Such release may substantially result from the co-administration of the GABA-a agonist and ARB (and/or ACE-I) dosages, but may, in certain embodiments be enabled or enhanced by co-administration of a dosage of EO-regulating compound. The step of increasing the release of vasodilatory compounds in systemic vessels may further encompass the increased release of EDRF stemming from co-administration of DOACs, arginase inhibitors, NO synthetase promoter(s). and/or AT2 agonist dosages.

This step of increasing vasodilating of relevant vessels by relaxation of smooth muscle to lower blood pressure and/or improve blood flow may result from the increased release of PGI2 and/or EDRF.

In some embodiments, for example, when DOACs are co-administered, an additional step of reducing bradykinin levels and/or clotting agents may occur and further contribute to the lowering of blood pressure and/or improvement of blood flow.

The step of treating (or preventing) the vascular disease or condition may proceed, in some circumstances with respect to the particular systemic circuit affected by the disease or condition. That is, increased PGI2 and/or EDRF may reduce blood pressure and/or increase blood flow in the targeted vessels, thereby improving the condition. eHTN may be treated, prevented, and/or improved by virtue of lowered blood pressure and/or improved blood flow in the systemic circuit and throughout the body. Bowel disease conditions may be treated, prevented, and/or improved by virtue of lowered blood pressure and/or improved blood flow in the mesenteric/splanchnic circuit. Microvascular diseases or conditions may be treated, prevented, and/or improved by virtue of lowered blood pressure and/or improved blood flow in peripheral microvascular vessels.

Certain metabolic diseases—including, for example, Diabetes Mellitus (“DM”)—may be characterized, at least in part, as micro-vessel disorders. Accordingly, certain metabolic diseases and conditions may be treated, prevented, and/or improved by virtue of lowered blood pressure and/or improved blood flow in the relevant microvasculature.

Consistent with the discussion of the GABA-a/AT1 construct and related biological mechanisms below, the co-administration of compounds discussed herein may also act similarly on diseased (or otherwise altered) higher value circuits in the human body, such as the cerebral, pulmonary, and kidney circuits—and ultimately result in lowered blood pressure and/or improved blood flow in those circuits. For example, it is contemplated that the co-administration of compounds discussed herein may improve a condition like pulmonary hypertension. Thus, the compounds, compositions, and methods of use discussed herein may also serve to treat or prevent certain diseases and deleterious conditions of, for example, the brain, lungs, and kidneys.

### Discovery of GABA-a/AT1 Construct and Related Biological Mechanisms and Beneficial Utilization Thereof

The co-administration of compounds as discussed herein may cause increased production and/or release of PGI2 via counter-intuitive biological mechanisms discovered by the inventor, as explained below. An increased release of PGI2 in systemic vessels may cause smooth muscle relaxation, which may, in turn, reduce blood pressure and improve blood flow in a wide range of systemic circuits.

The inventor has discovered that a GABA-a receptor with activity on peripheral pregnant systemic vascular smooth muscle and/or endothelium may be modulated by the presence or absence of AII binding to an associated AT1 receptor to induce increased release of PGI2. Moreover, the inventor discovered that the binding both GABA-a agonist and an ARB to that GABA-a/AT1 construct may induce net PGI2 formation in systemic vessels in humans with eHTN or an otherwise diseased vasculature.

Consequently, the inventor has determined that preventing AII from binding to AT1 receptors permissively supports increased release of PGI2 in various vascular circuits—including in the systemic, mesenteric, and peripheral microvascular—and compartments, in the presence of GABA-a agonists. The PGI2 released from both endothelium and smooth muscle of the effected circuits may increase vasodilation and preferential flow to that vascular bed, and enhance the net level of blood flow and distribution throughout the entire vascular system.

During experimentation between uterine vessels and systemic vessels during pregnancy, an inverse response between uterine and systemic vessels and their levels of PGI2 release was found. Pregnant systemic vessels exposed to GABA, and in the absence of angiotensin II, showed increased PGI2 release. Pregnant uterine vessels, however, required GABA and angiotensin II to increase PGI2. The inventor has surmised that this mechanism is likely feto-productive in cases of hemorrhage and low blood volume during pregnancy. During these times, AII increases, dilating the uterine circuit and constricting the systemic circuit, thereby allowing the maternal blood to pass by the baby, prolonging the baby's chances of survival.

The inventor has surmised that this presumably feto-protective mechanism is initially present in every human, but disappears shortly after birth, then reappears in adult when certain cardiovascular states come about—notably pregnancy and diseased vasculature. More specifically, the inventor believes that biological mechanism is re-expressed in affected vascular systems in adulthood. There are fetal genes at play in pregnancy, and different responses in the maternal circuit to angiotensin II and endogenous ouabain vis-à-vis the systemic circuit: For example, fetal hemoglobin will revert to adult hemoglobin after 1 year of life; and, in disease states such as preeclampsia, the blunted effects to angiotensin II or EO become more pronounced. Thus, the co-administration of disclosed compounds and compositions thereof may serve to relax smooth muscle of affected vascular systems to improve cardiovascular-related diseases, conditions, and symptoms discussed herein.

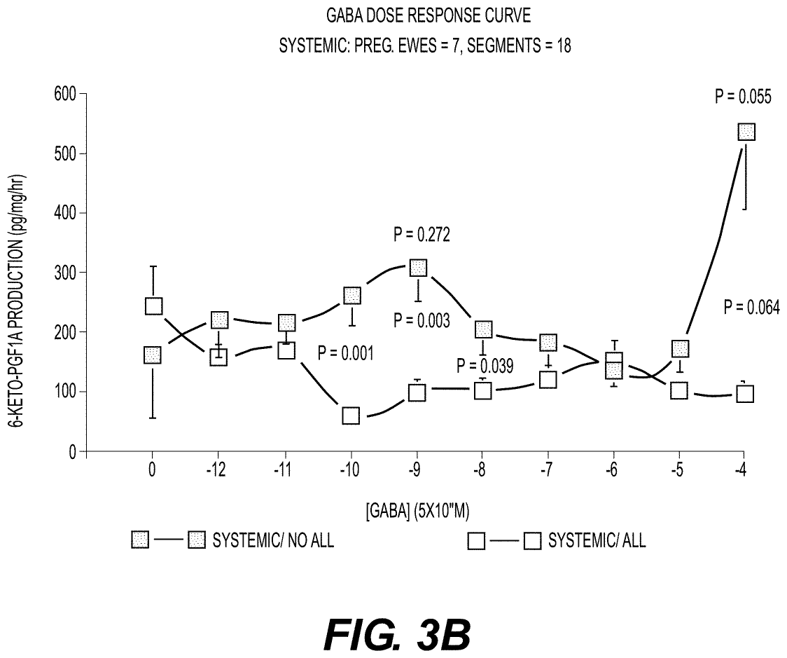

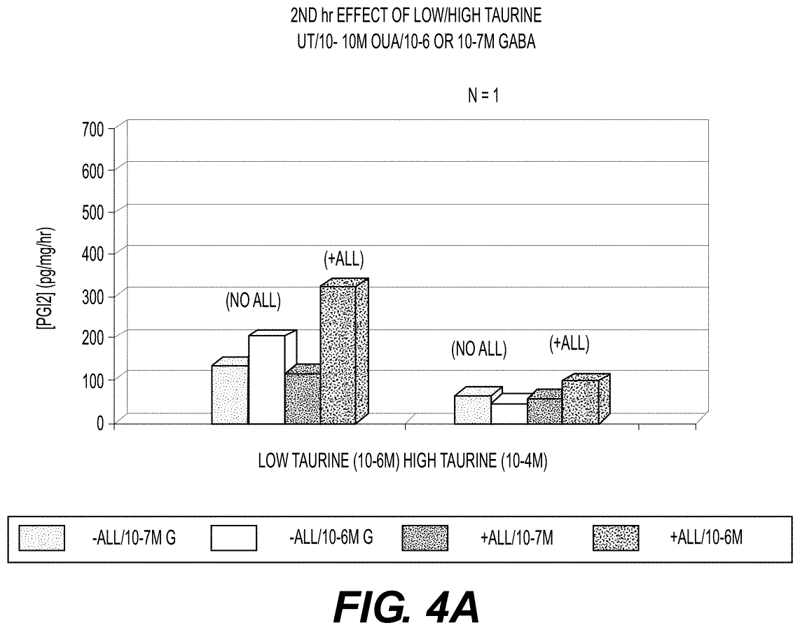

With reference to FIGS. 3A and 3B, the inventor's laboratory data indicates that release levels of PGI2 vary with the presence of AII and GABA levels—and are highly dependent on the vascular circuit. As shown in FIG. 3A, in the pregnant uterine circuit, more PGI2 is released in the presence of AII than without the presence of AII—at most levels of GABA. It may further be noted that GABA concentrations of −7 M, −6 M, and −5 M—which correspond to GABA concentrations in the plasm, VSM, and endothelium, respectively—resulted in no significant PGI2 release difference responsive to the presence or absence of AII. As shown in FIG. 3B, the relationship between PGI2release and AII presence is substantially inverted in the pregnant systemic circuit. Accordingly, as may be observed, there is an inverse relationship depending on the vascular circuit.

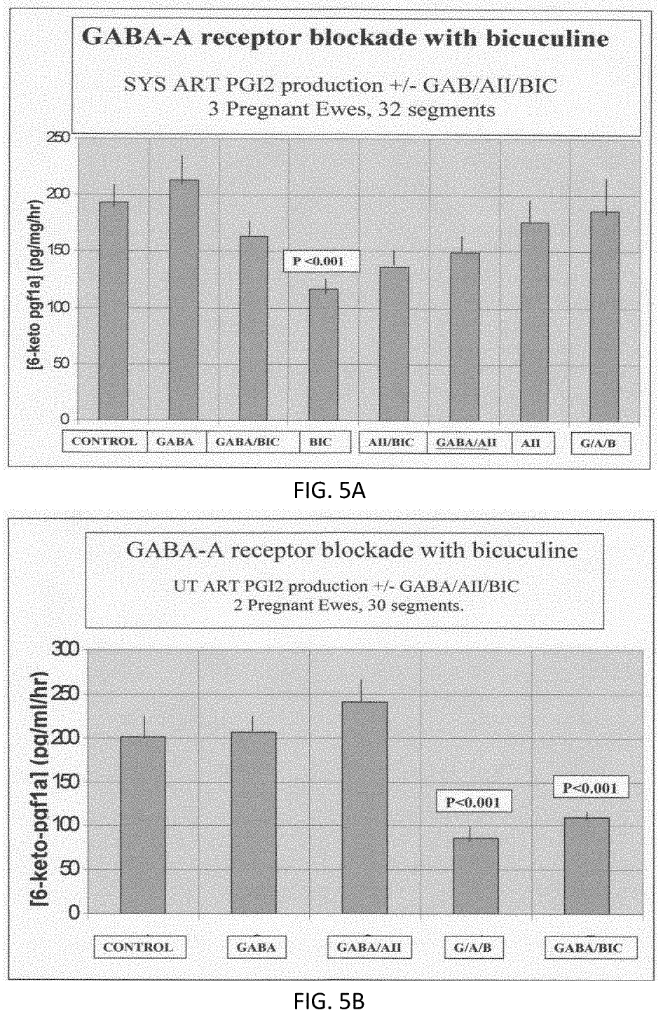

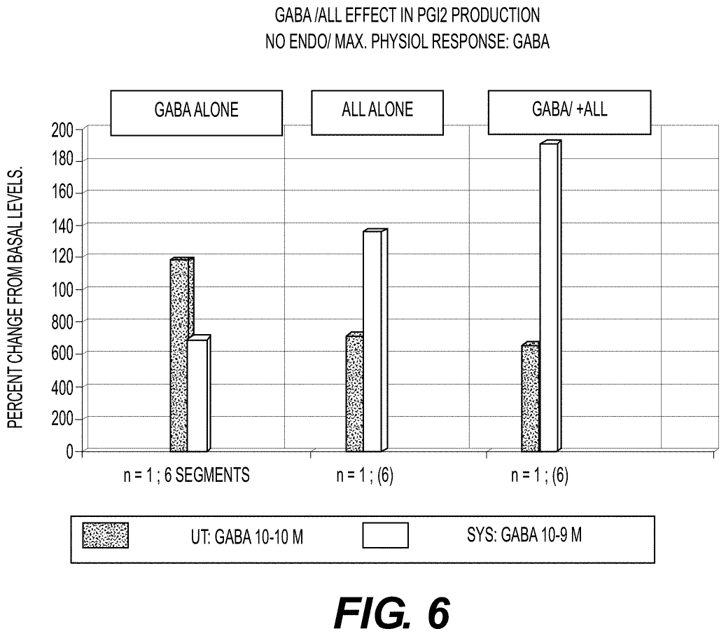

With reference to FIGS. 5A and 5B, the inventor's laboratory data indicates the presence of GABA-a receptors in both systemic vessels and uterine vessel in pregnant animals. In this experiment, the inventor blocked the GABA-a receptors with bicuculine, which reduced PGI2 release in both circuits. It may be noted that the third and last columns of FIG. 5A and the last two columns of FIG. 5B reflect competition on the GABA-a receptor between GABA and bicuculine. GABA improves PGI2 response substantially more in the absence of GABA-a receptor competition (e.g., bicuculine); and GABA with bicuculine improves PGI2 response substantially more than bicuculine alone.

With reference to FIG. 5A, the inventor observed that pregnant systemic vessels responded to make PGI2, but only in the absence of AII binding (here, exemplified by a lack of AII) and when GABA-a receptor was stimulated.

With reference to FIG. 5B, the inventor observed that in pregnant uterine arteries, the blocking of GABA-a receptors with bicuculine substantially reduced PGI2 release, even when additional GABA was added (col. 5); the further addition of AII resulted in even further decreased PGI2 (col. 4). The addition of GABA had an insignificant effect on PGI2 release (col. 2) and the addition of GABA and AII increased PGI2 release (col. 1).

Via similar experiments using phaclofen (results not shown), a selective GABA-b antagonist, (i.e., instead of bicuculine, a selective GABA-a antagonist), the inventor found that the GABA-b receptor did not affect PGI2 release (data not shown). Accordingly, the inventor determined that the GABA-a receptor was the relevant GABA receptor for the GABA-a/AT1 construct. Thus, a GABA-a agonist is sufficient to meet the GABA action requirements for exploitation of the GABA-a/AT1 construct.

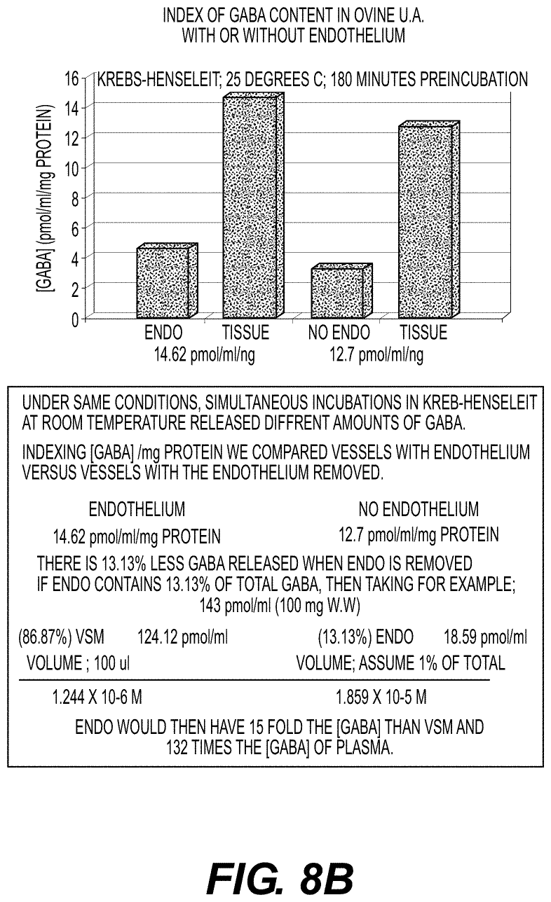

With reference to FIGS. 8A and 8B, the inventor's laboratory data indicates that GABA concentrates by 1 and 2 orders of magnitude—from plasma, to smooth muscle, to endothelium. More specifically, FIGS. 8A and 8B collectively illustrate measurements of GABA concentration from vascular tissues, both with and without the endothelium. The GABA concentrations were different, and, taking into account the single cell layer of endothelium, the GABA concentration difference was calculated to be almost 10× higher in the endothelium than the smooth muscle.

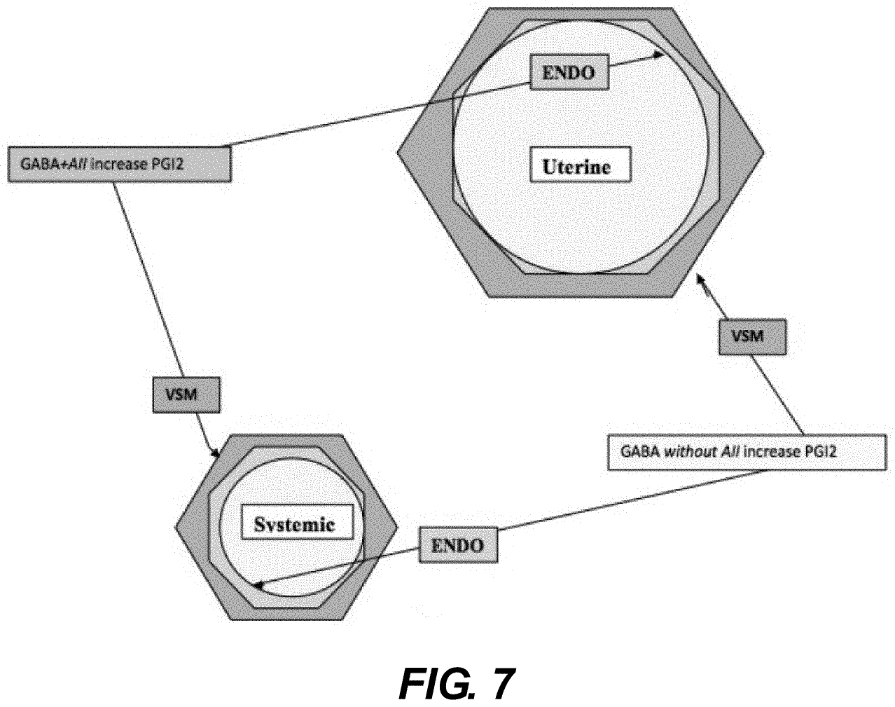

With reference to FIG. 6, the inventor's laboratory data further illustrates that when the endothelium is removed from the pregnant uterine and systemic vessels, respectively, the PGI2 release trends that had been seen in the intact vessels is reversed. As may be observed, PGI2 release in pregnant uterine vessels is reduced in the presence of AII when the endothelium is removed; inversely, PGI2 release in pregnant systemic vessels is increased in the presence of AII when the endothelium is removed.

In view of the data and descriptions of FIGS. 8A, 8B, and 6, the inventor's laboratory data illustrates the unexpected result that PGI2 formation is affected by the variable concentrations of GABA (acting on GABA-a receptors) within different compartments of the uterine artery. The rise in PGI2 release per compartment is directly related to increasing concentration of GABA. Depending on the vascular circuit, the GABA-a mediated PGI2 increase is a function of the presence or absence of AII. In pregnancy, this appears to be a protective mechanism to support blood flow to the baby in hypovolemic states.

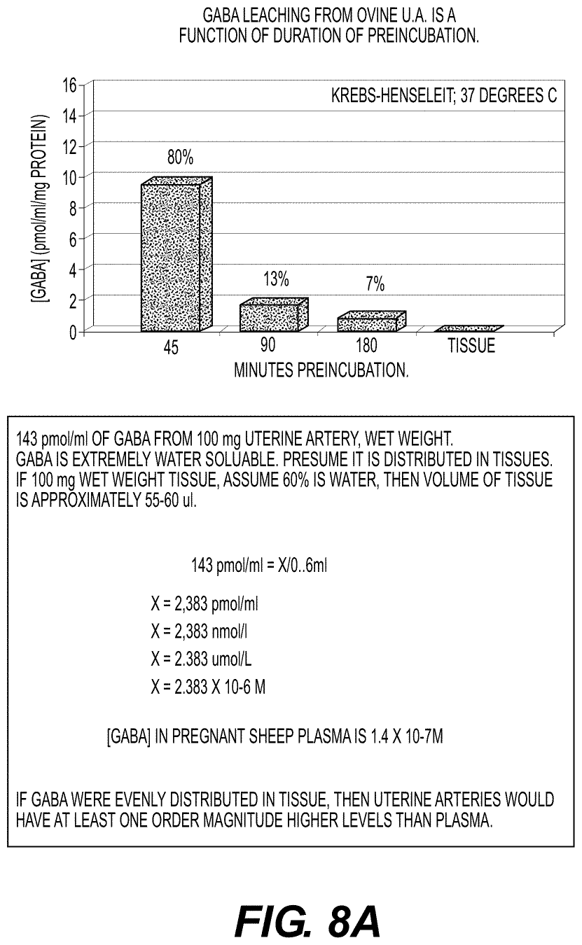

In view of the above, the inventor surmised that, during pregnancy, in the endothelium of systemic vessels and in the vascular smooth muscle (“VSM”) of uterine vessels, PGI2 secretions increase in the presence of GABA-a agonists and the absence of AII. Conversely, the inventor discovered that, during pregnancy, in the vascular smooth muscle of systemic vessels and in the endothelium of uterine vessels, PGI2 secretions increase in the presence of GABA-a agonists and AII. Thus, as illustrated in FIG. 7, there is a paradoxical inverted effect on different vascular circuits/compartments in PGI2 formation due to the action of GABA-a agonist and AII. In other words, the PGI2 release effect is variable between the smooth muscle and endothelial cell compartments, suggesting a mechanism of variable net PGI2 production and smooth muscle relaxation.

### Ouabain-Taurine relationship to GABA-a/AT1 Construct

Taurine is a sulfur amino acid, but is not considered to be an essential amino acid as it is plentiful in foods. Taurine homeostasis may be maintained in the body via ingestion and renal/fecal excretion. De novo metabolism may occur in the brain and the liver, but only in small amounts. During the menstrual cycle, taurine may be actively transported into the uterine lumen to exceed concentrations four hundred times that of the next highest amino acid. This compound is 2-amino-ethane sulfonic acid.

In non-pregnant humans, Taurine is generally considered to be ubiquitous in the blood stream. In pregnant humans, the maternal compartment is generally characterized as having low Taurine and the fetal compartment is generally characterized as having high Taurine.

Although Taurine has been studied for over 60 years, its function in the human body largely remains unclear. However, Taurine is known to be able bind to the insulin receptor and displace insulin, which may induce hypoglycemia in certain circumstances. It is an important amino acid in normal fetal growth and development and fetal deprivation produces growth retardation, mental deficiency, and seizures. At low concentrations, Taurine may bind to and activate the GABA-a receptor. However, at higher concentrations, it may competitively inhibit the same receptor. That is, Taurine may act as a GABA-a antagonist. Other evidence suggests Taurine may bind to the GABA-a receptor and allosterically modify it. In view of these varied and seemingly contradictory teaching, it appears that there may be an interplay between Taurine and GABA in pregnancy. Such interplay may also play a role in the treatment and prevention of other disorders that relate to the GABA-a/AT1 construct, including eHTN, ischemic bowel disorders, peripheral vascular insufficiency, microangiopathic disease associated with DM, and other vascular-related maladies discussed herein.

During pregnancy, active renal taurine excretion may be selectively inhibited, and active placental uptake may occur via Na/K ATPase transport mechanisms. This uptake may provide a fetus with the Taurine it needs to grow; the corresponding feto-maternal gradient may be greater than a 1000 to 1. Taurine may be found throughout the body, but in large amounts in the heart, striated muscle, and certain parts of the brain. Additionally, platelets and white blood cells (WBC) may have active transport systems to generate high intracellular gradients, typically greater than 400/1.

Peripherally, intravascularly infused Taurine is understood to have no cardiovascular effect in normal non-pregnant animals. While Taurine has been shown to lower blood pressure initially, over two weeks infusion, taurine raises blood pressure. In studies on spontaneously hypertensive rats and SHR-prone rats, the supplementation of taurine, either through diet, parenterally, or intravenously prevented or delayed the onset of hypertensive sequellae, and further prevented strokes in those suspectable rats. Centrally, Taurine injected intra-ventricularly can block the effects of II, thereby dampening the AII adrenergic augmentation typically observed in rats.

Endogenous Ouabain (“EO”) is putative endogenous cardiac glycoside, and was identified in the 1980s. Endogenous ouabain is higher in pregnancy than in the normotensive state; and it is higher in preeclampsia than in normal pregnancy. Although EO has been associated with pregnancy and essential hypertension, its biological mechanisms are far from settled in the literature. Currently the origin of circulating EO is believed to be the adrenal system, but other sources have been suspected.

The prior art has noted certain paradoxical effects of EO in pregnancy and preeclampsia. For example, in Endogenous Ouabain: Recent Advances and Controversies, Dr. Hamyn noted the following: (i)“[N]ormal pregnancy is a high EO state with apparent resistance of the arteries to the pressor action of circulating EO. Indeed, even supra-physiological circulating levels of ouabain failed to raise BP in pregnancy.”; (ii) “Circulating EO is linearly related to BP in preeclampsia, suggesting that the mechanism underlying ouabain resistance is impaired so that the already elevated EO could raise BP in a dose-dependent manner.”; (iii) “Thus, in this preeclamptic model in which EO is believed to be elevated, ouabain behaved as an antihypertensive and had a net effect on BP that resembled that of digoxin in ouabain-dependent hypertension. The mechanism of this paradoxical and beneficial effect requires investigation. Nevertheless, it now appears that, contrary to earlier ideas, EO upregulation in preeclampsia is of potential benefit to mother and fetus.”; (iv) “At the opposite end of the pregnancy spectrum, recent studies link low circulating EO levels with impaired fetal growth and development. . . . Ouabain is recognized as a growth promoter, but these new results are the first to suggest that relative lack of EO increases the risk for impaired fetal development. In this context, the aforementioned ouabain resistance of pregnancy makes sense: the elevated circulating EO could exert a growth promoting effect while its hypertensinogenic activity was deactivated.” (Id.)

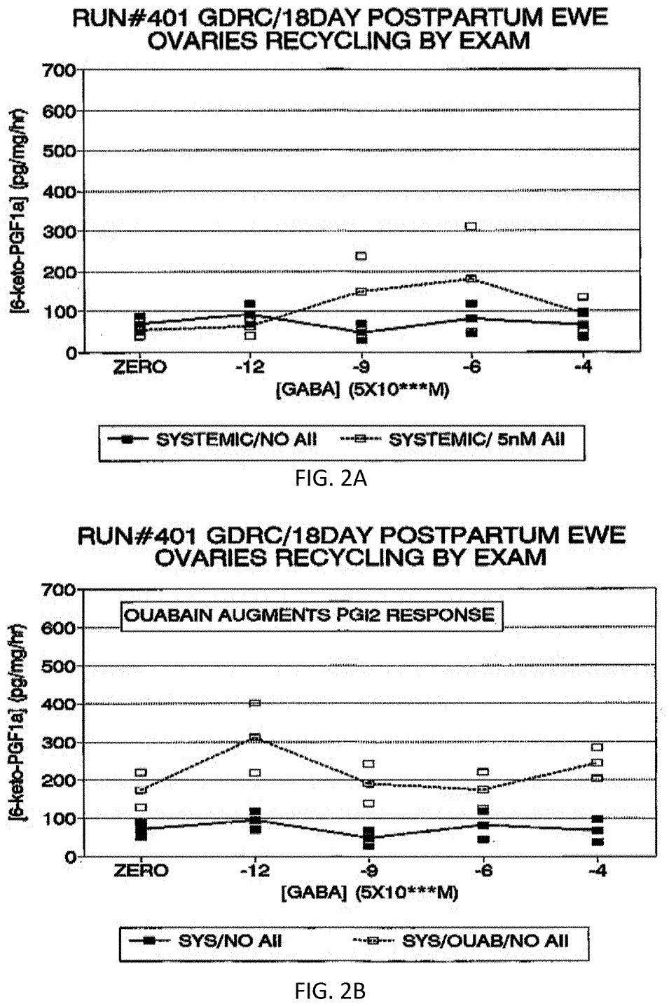

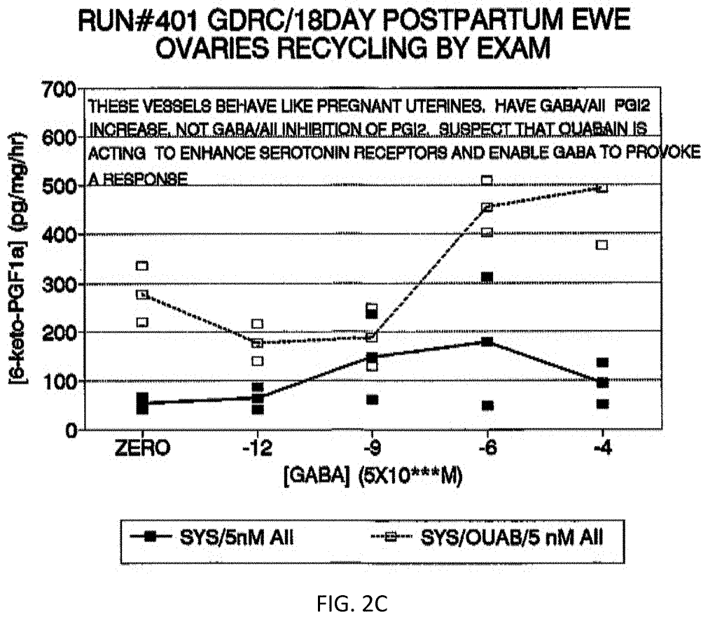

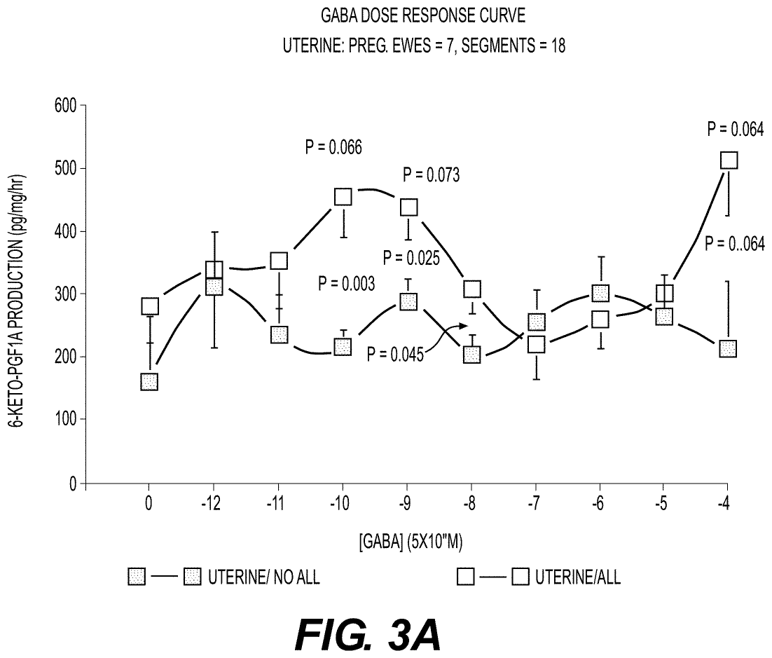

As discussed above, a version of the GABA-a/AT1 construct is present in pregnant uterine tissues, which are also generally characterized as having low dose Taurine. Normotensive non-pregnant systemic vessels possess the same version GABA-a/AT1 construct, but are generally characterized as having high dose Taurine. The inventor has determined that the presence of at least low dose Taurine appears to be important to GABA-a/AT1 construct's inducement of PGI2 release. Further, with reference to FIGS. 2A-2C discussed below, the inventor has determined that EO modulates the basal output of PGI2 via the GABA-a/AT1 construct in systemic non-pregnant vessels. This has not been noted in the literature for systemic vessels, whether pregnant or non-pregnant. That the Ouabain exerts an impact on the PGI2 modulation in these non-pregnant normotensive vessels, suggests the construct is present but lacks the EO conditions of pregnancy, PIH, or eHTN to become active.

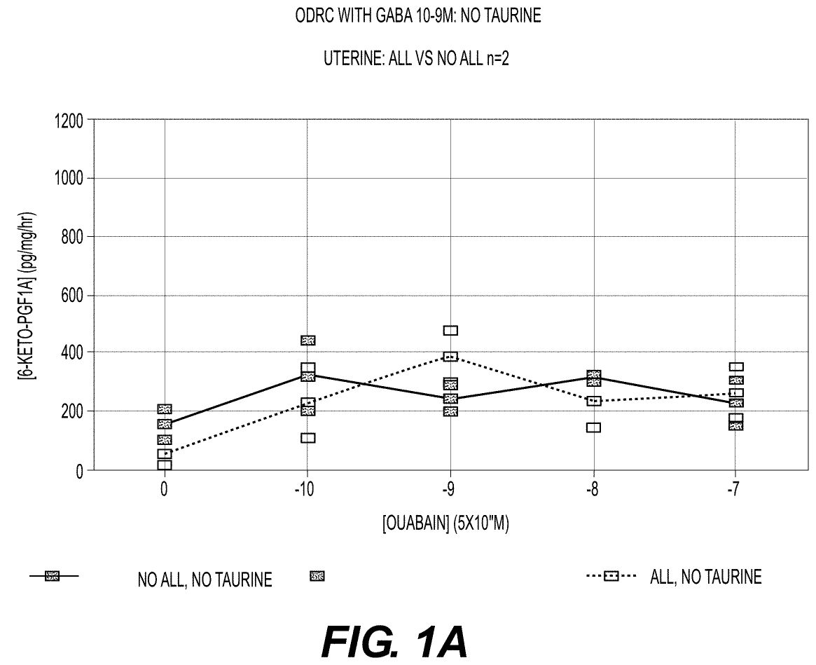

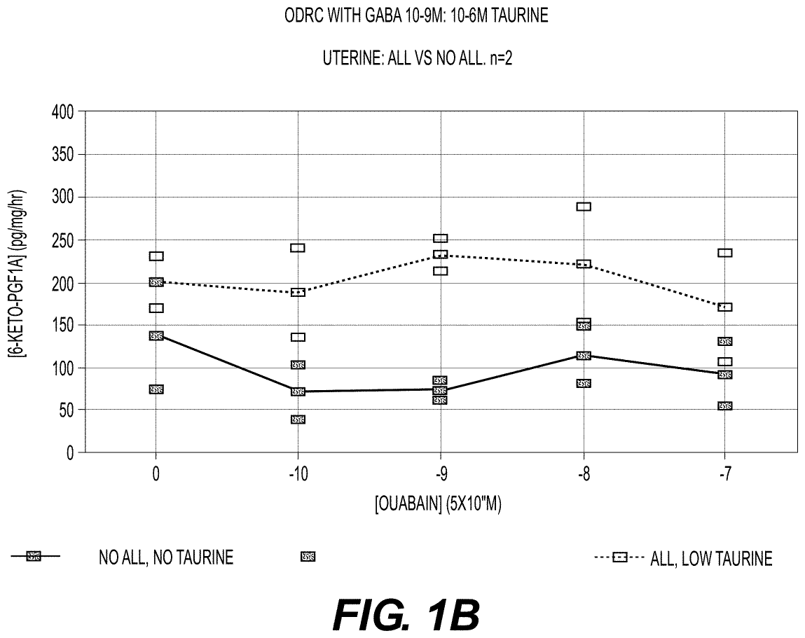

FIG. 1A illustrates an Ouabain Dose Response Curve (“ODRC”) in pregnant uterine tissue. With reference to FIG. 1A, the inventor has observed that without taurine and at various levels of EO, the presence or absence of AII has negligible effect on PGI2 release in pregnant uterine arteries. This may indicate inactivity of the GABA-a/AT1 construct without Taurine. It may have been expected that substantially less PGI2 would be released without AII than with AII. However, such effect was not observed until low dose Taurine was provided, as demonstrated in FIG. 1B. This may indicate an active GABA-a/AT1 construct under such conditions. It may also be noted that, regardless of AII presence, overall PGI2 release appears higher without Taurine (e.g., FIG. 1A) that with Taurine; this may be explained by Taurine's GABA-a antagonizing characteristics. Accordingly, the inventor has surmised that non-pregnant normotensive systemic vessel behave similarly to pregnant uterine vessels when exposed to Ouabain, releasing higher levels of PGI2 in the presence of GABA and AII.

Notably, a study performed by another researcher indicated that the presence or absence of AII did not affect PGI2 response in nonpregnant, normotensive systemic or uterine vessels. While FIG. 1A tracks these prior art results, FIG. 1B illustrates an unexpected divergence in the presence of Taurine.

Thus, it appears that Taurine may be needed (or, at a minumum, may be substantially helpful) for PGI2 formation/release. It is believed that low dose taurine may upregulate GABA-a receptors, and this may be connected to PGI2 formation and release.

In FIGS. 2A-2C, GABA dose response curves of systemic vessels in 18 day postpartum sheep are provided. These vessels are believed to be representative of non-pregnant normotensive systemic vessels. With reference to FIG. 2A, no EO or Taurine was provided, and the resulting of a dose response curve for systemic vessels illustrates no PGI2 response, as might have been expected from the prior art. Accordingly, it appears that at least one of Taurine or EO (or the like) may be needed to up-regulate the GABA-a receptor to augment PGI2 levels in the presence of nanomolar ouabain, or may at least be substantially helpful to increase PGI2 levels. Thus, on 18 day postpartum sheep, PGI release/production by intact systemic vessels exposed to GABA and without Taurine or EO, shows no significant PGI2 formation by the presence or absence of Angiotensin II.

With reference to FIG. 2B, the addition of Nanomolar Ouabain showed a significant increase in PGI2 levels at most concentrations of GABA and without AII, on intact nonpregnant systemic vessels. The prior art does not disclose or suggest such a PGI2 response to EO in systemic vessels, either with or without Angiotensin II. Such results may be considered unexpected and even parodoxical because, as shown in FIG. 2B, without Oubain, the nonpregnant systemic vessel did not release significant PGI2 with GABA. Because this response is similar to the response in the pregnant uterine vessels (see, e.g., FIG. 1B), this suggests that the normotensive nonpregnant systemic vessels manifest a similar PGI2 control mechanism as pregnant uterine vessels.

With reference to FIG. 2C, the addition of Nanomolar Ouabain showed a significant increase in PGI2 levels at most concentrations of GABA and with AII. In FIG. 2C, the systemic vessels react to variable GABA in a similar manner as pregnant uterine vessels (see, e.g., FIG. 1B), but only in the presence of Ouabain.

By comparing FIGS. 2B and 2C, it may be observed that in the presence of Ouabain, the addition of AII leads to substantially more PGI2 release in normotensive nonpregnant systemic vessel, especially at higher concentrations of GABA. Accordingly, the inventor has unexpectedly discovered that Ouabain exposure may modulate the PGI2 response through interaction with the GABA-a/AT1 construct.

It may be noted that responses in FIGS. 1A and 1B are substantially similar to FIGS. 2A and 2B/2C (Ouabain datapoints), respectively, with respect to Ouabain, GABA, and AII responses. Accordingly, the inventor has observed that pregnant uterine arteries may mirror non-pregnant, normotensive systemic arteries in relevant respects. That is, the inventor believes that the pregnant uterine vessel state for GABA-a/AT1 receptor construct is the default state for normotensive, healthy systemic vessels outside of pregnancy. Conversely, the inventor believes that the pregnant systemic vessel state for GABA-a/AT1 receptor construct is the default state for hypertensive or otherwise diseased systemic vessels outside of pregnancy.

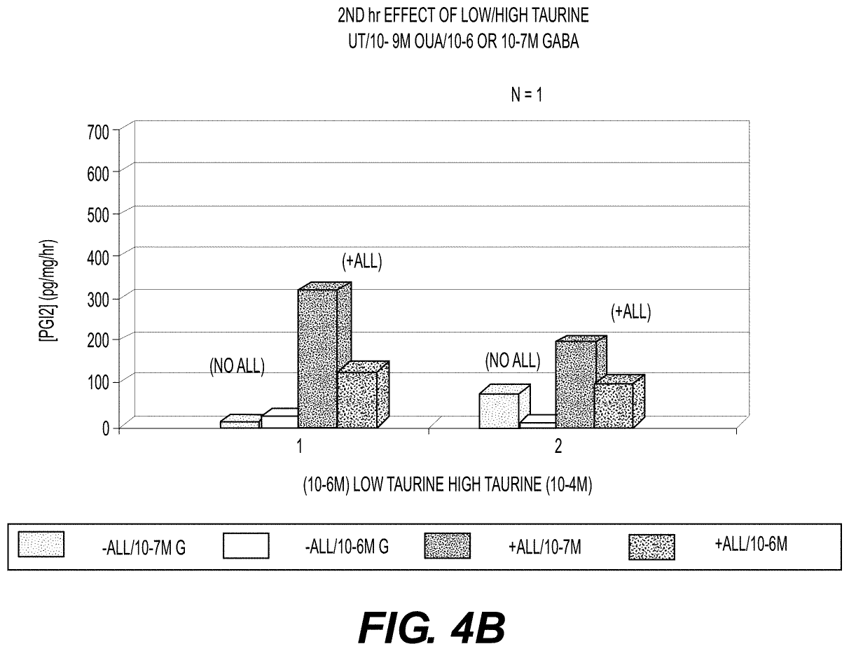

With reference to FIGS. 4A and 4B, the biological mechanism is further illustrated: Pregnant uterine vessels demonstrated higher PGI2 release with an increase in Ouabain from −10 M to −9 M, at low taurine conditions (e.g., not fetal compartment), and in the presence of physiological GABA (e.g., at approximate −7 M) concentrations. This increase in PGI2 release in the presence of higher Ouabain may explain the EO resistance noted in pregnancy. Moreover, the increase in PGI2 release in the presence of higher EO levels, as shown in FIGS. 4A and 4B, indicates that in some circumstances, stimulating EO release, providing Ouabain or Ouabain-like substances, and/or providing substances to upregulate Ouabain's effects may enhance or enable exploitation of the GABA-a/AT1 construct to produce PGI2.

FIGS. 4A and 4B illustrate results at low and high taurine to represent the maternal and fetal compartments, respectively. At low levels of taurine (e.g., maternal compartment) and low Ouabain, PGI2 release increased with GABA and AII. However, when Ouabain was increased by one order magnitude, PGI2 release increases in the presence of AII became significant at lower levels of GABA at low levels of Taurine (e.g., maternal compartment), and were even observable at high levels of Taurine (e.g., fetal compartment).

In view of the above, the inventor has discovered the interplay of GABA-a receptor with AII activity may be regulated by Ouabain to promote and augment PGI2 release and/or production. Such discovery partially explains the EO resistance of pregnancy, and how the disruption of this PGI2 mechanism may contribute to preeclampsia and eHTN.

The literature provides that EO increases in pregnancy and, even more so in preeclampsia (“PIH”) and eHTN. Despite this, the literature has been unable to make sense of the paradoxical effect of EO between these states. In normotensive pregnancy, both AII and EO have a blunted pressor response, and this appears to be lost in preeclampsia (PIH). The inventor has surmised that during eHTN and other states, the systemic vessels revert back to the pregnant systemic vessel receptor construct, but this time without the placental GABA source to drive PGI2 production. EO and AII have both tend to increase blood pressure in non-pregnant patients: In accordance with the discussion and results above, it may be surmised that they are related via biased receptor signaling at the GABA-a/AT1 complex.

### AT2 Agonists

The ultimate vasodilatory effect of increasing PGI2 may be synergically improved by simultaneously increasing EDRF release. In this manner, blood pressure and blood flow characteristics may be improved by pairing these two complementary, vasodilatory biological mechanisms—namely, increased release of PGI2 through the GABA-a/AT1 construct and increased release of EDRF through the AT2 receptor.

In the art, C21, an AT2 agonist, has not shown to reduce blood pressure in eHTN by itself. C21 has, however, done so in combination with of a non-blood pressure reducing ARB.

Saralasin and Sarile are additional examples of AT2 agonists. These substances are also considered ARBs and, accordingly, their use in a treatment composition or regimen may be particularly advantageous.

By utilizing the GABA-a/AT1 construct in concert with C21 and/or other AT2 agonist(s), both PGI2 and NO may be promoted together, and accordingly may further augment vasodilation, reduce blood pressure, improve blood flow, and/or otherwise improve the function and operation of various vascular circuits.

### Expression and Utilization of GABA-a/AT1 Construct in eHTN and other Patients

As noted above, the inventor has surmised that non-pregnant normotensive (and otherwise healthy) systemic vessels may mirror pregnant uterine arteries with respect to the GABA-a/AT1 construct. That is, PGI2 release may be induced by AII binding to the ATI receptor, and such release may be increased by GABA-a receptor binding to an agonist. EO and/or Taurine levels may further regulate this biological mechanism.

The inventor has discovered that eHTN and preeclampsia may be treated via the same process of vasomotor control for blood pressure regulation, arising from the presence of the peripheral vascular endothelial GABA-a/AT1 construct in pregnancy and subsequent loss of activity after delivery. In pregnancy, the above-described GABA-a/AT1 construct is a backup protective system of preferential vascular flow in hypovolemic situations, to shunt blood flow to higher class circuits such as the uterine vessels (baby), lungs, kidneys, and brain.

Accordingly, the inventor has surmised that in the systemic vessels of adult (non-pregnant) eHTN and other disease states, the GABA-a/AT1 construct of pregnant systemic vessels is effectively re-expressed. In this state, PGI2 release is increased by the blockage of the AT1 receptor (or absence of AII) and presence of GABA-a agonist(s). However, unlike in pregnancy, the non-pregnant (diseased) systemic vessels lack a placental GABA source to bind to the GABA-a receptor. And without the GABA-producing placental conditions of pregnancy, the GABA-a/AT1 construct is unable to cause the release of sufficient PGI2.

Accordingly, the provision of an ARB (and/or ACE-I) and a GABA-a agonist to an eHTN patient (or a non-pregnant patient with similarly diseased systemic vessels) has been determined to increase PGI2 release in non-pregnant systemic vessels, particularly in the endothelium. The synergistic, PGI2-releasing effect of ARB (and/or ACE-I) and GABA-a agonist results in blood pressure reduction and blood flow improvements far greater than what the expected result of administering the same dosage of an ARB and the same dosage of a GABA or GABA-a agonist would be.

The inventor has further surmised the higher order circuits typically mirror the biological mechanism of non-pregnant normotensive (and otherwise healthy) systemic vessels, which in turn mirrors those of pregnant uterine arteries. These higher order circuits may continue to do so even where the (non-pregnant) patient's systemic vessels are afflicted by eHTN or other conditions which reverse the effect of AII binding to the AT1 receptor on the GABA-a receptor in those systemic vessels.

In view of the above discussion relating to EO, PGI2 release/production may be further promoted via the providing a GABA-a agonist and ARB (and/or ACE-I) in conjunction with one or more compounds that regulates EO up or down. In this application, substances that stimulate or hinder EO release, Ouabain or Ouabain-like substances, and/or substances to upregulate or downregulate Ouabain's effects may be collectively referred to as EO-regulating compounds. As a first class of EO-regulating compounds, Digoxin, digitalis, digitoxin, β-acetyldigoxin and methyldigoxin and similar compounds may antagonize EO's effects by binding the EO receptor and blocking EO action. As a second class of EO-regulating compounds, low dose glucocorticoids or other Glycoside antagonists may lower EO levels. As a third class of EO-regulating compounds, diuretic HCTZ and other salt-depleting substances may effectively increase EO by stimulating EO release. As a fourth class of EO-regulating compounds, providing Nanomolar Ouabain or an Ouabain-like substance may, in effect, achieve results akin to an increase of EO. Accordingly, in some embodiments, adding one or more EO-regulating compounds to a GABA-a agonist/ARB dose combination or the like may enhance basal PGI2 output.

In some embodiments, it is contemplated that a patient's EO levels may be tested prior to prescription and/or administration of ARB/ACE-I and GABA-a agonist dosages. Following such testing, it may be determined with a patient's EO levels are in an optimal range for increased PGI2 release or should be regulated either down or up. Based on such determination, no EO-regulating compounds may be provided, one or more members of the first and/or second classes of EO-regulating compounds may be provided to up-regulate Ouabain effects, or one or more members of the third and/or fourth classes of EO-regulating compounds may be provided to up-regulate Ouabain effects.

Optimal, excessive, and/or insufficient EO levels in patients—as well as effectiveness each EO-regulating compound—may be experimentally determined by a person of ordinary skill in the art and may depend on a particular medical condition and/or the particular compounds administered.

### Bowel Disorder Treatment and Prevention

While the exploitation of GABA-a/AT1 construct and other biological mechanisms have been heretofore described primarily in terms of treating eHTN via increasing PGI2 and/or EDRF in systemic vessels, the same or substantially similar compounds and composition may serve to improve blood flow and reduce blood pressure in various other vascular circuits.

Disease conditions affecting particular systemic circuits may stem from or otherwise substantially relate to blood flow or blood pressure problems within the relevant circuits. Accordingly, exploitation of GABA-a/AT1 construct and other biological mechanisms within affected circuits may serve to treat or prevent relevant disease conditions in whole or in part. For example, nonpregnant mesenteric/splanchnic and peripheral microvascular vessels are believed to behave similarly as other systemic vessels in the presence of a GABA-a agonist and ARBs. Thus, co-administration of such substances may be utilized treat IBS/IBD and peripheral microvascular disease to improve blood flow in affected vascular circuit flow.

Blood flow to and through the splanchnic/mesenteric vessels is associated with disorders such as Irritable bowel syndrome (e.g., type C or D, or mixed); Inflammatory bowel disease (IBD), including ulcerative colitis (UC), regional ileitis, and crohn's disease; and/or the like.

These inflammatory diseases are considered to be autoimmune in nature and have been treated with newer monoclonal blocking antibodies to TNF alpha. Older approaches included providing a patient with an aspirin-like compound, a low dose allopurinol, a gout lowering medication, or 5-flurouracil. During the 1990s, it was noted that their IBD symptoms improved and often resolved while on the heparin (and/or lovenox). However, this treatment regimen has since lost favor due to the inconvenience of administering twice daily injections and requisite monitoring to ensure that the patient does not suffer from excessive anticlotting effects. The heparin/lovenox effect may be driving the NOS to NO pathway in the bowel, allowing improved mesenteric perfusion and tissue repair.

Any organ perfused with oxygenated blood has at least three major required roles. Using the heart as an initial example, the first role from perfusion is to keep itself alive via the coronary arteries. The second is the functional role of this organ. With respect to the heart, the function is to drive blood to the tissues and keep the rest of the body alive. The third role is to have extra capacity to allow for locomotion/activity. If there is restricted flow to the heart, perhaps only two of three roles may be met. If one is at rest, then the heart and person remain functional, and no chest pain would be expected. If however, a person afflicted with restricted flow to the heart engages in strenuous physical activity (e.g., attempting to run upstairs), the locomotion and diversion of oxygenated blood may create injury to another organ, as a steal syndrome, or angina—perhaps leading to heart attack.

The bowel is similar in the three major tasks it must complete. The first is to maintain itself and repair itself. Failure to do so may lead to ulceration, bowel dysfunction, thrombogenic ischemia, transmural fibrosis, and/or lesions. The second role is to absorb nutrients; failure to do so may result in diarrhea and malabsorption. The third role is the peristalsis or locomotion to move waste through the GI tract. With decreased mesenteric/splanchnic perfusion, perhaps only two of three roles may be met; this may lead to constipation, diarrhea, malabsorption, and/or other maladies. Ultimately, this may lead to the breakdown of the luminal barrier due to enzymes and/or bacterial infections, and may manifest as IBD with concomitant immunological responses, bleeding, and/or obstruction.

The exploitation of the GABA-a/AT1 construct to drive PGI2 vasodilation in a diseased mesenteric/splanchnic circuit may augment blood flow to improve performance of the three roles, thereby improving overall function. This could be done with oral compositions disclosed herein, which may have many advantages over the current standard of care, for example, the 2×/day heparin or lovenox injections and/or oral antifactor Xa agents, which may increase NO activity via a distinct biological mechanism.

Bowel disorders such as IBS and IBD have been considered multi-factoral. Intestinal ischemia may play a substantial role in the pathogenesis of necrotizing enterocolitis (NEC). Intestinal vascular resistance may be determined by a dynamic balance between vasoconstrictive and vasodilatory inputs. In a newborn, this dynamic balance heavily favors vasodilation secondary to the copious production of endothelium-derived nitric oxide (NO); this circumstance may serve to ensure adequate blood flow and, thus, oxygen delivery to the rapidly growing intestine. Endothelial cell injury may shift this balance in favor of endothelin (ET)-1-mediated vasoconstriction, which may lead to intestinal ischemia and tissue injury. In view of the fact that coagulation necrosis may occur in the majority of neonatal necrotizing enterocolitis (NEC) patients, intestinal ischemia is believed to be a substantial contributing factor to the pathogenesis of NEC.

Mesenteric arterial-necrosis (MA) occurs in horses as colic, as a natural endotoxemia (ET), and this can be devastating. The horse model is not unlike neonatal NEC. Accordingly, the pathogenesis of the MA may be fundamentally similar in the two groups—and MA may be a pathognomonic feature of equine ET. Similar changes have been observed in cases of prolonged angiospasm or vasoconstriction. The effects of sustained arterial contraction may lead to intimal and medial damage influence the pathomorphogenesis of MA, which may display a similar transmural pathology to crohn's disease. That ischemic bowel disorder is driven by a myriad of sources (e.g., infectious-endotoxic and/or ischemic/reperfusion damage) suggests that abnormal mesenteric perfusion is a hallmark of these related diseases.

In view of the above, the inventor has determined that the GABA-a/AT1 construct may be readily utilized to treat certain bowel disorders. More specifically, the inventor has surmised that in adult (non-pregnant) splanchnic/mesenteric vessels in disease states, the GABA-a/AT1 construct of pregnant systemic vessels is effectively re-expressed. The exploitation of such biological mechanism as described above may increase PGI2 release, resulting in increased vessel dilation within the affected mesenteric/splanchnic vessels. Such dilation may improve blood flow and, accordingly, oxygenation and functioning of the bowel, improving various related disease conditions.

### Peripheral Vascular Disease

The systemic vessels outside of the mesenteric/splanchnic circuit, which may be referred to as the peripheral systemic vascular circuit, may include the skeletal muscle and distal extremity vascularity circuit. Diminished vessel diameter in such circuit(s) may manifest as microvascular peripheral vascular disease (“PVD”). This may present as systemic sclerosis, vascular insufficiency, and distal ischemia—all of which are notably associated with COVID-19 infection and/or sepsis.

Inflammation and PVD is associated with clotting imbalance, with higher thrombophilia and reduced fibrinolysis. Bradykinin is associated with an increase in sympathoadrenal effects of ‘new pressor protein’ related to human blood coagulation factor XII fragment. In contrast, thrombin generated during disseminated intravascular coagulation in rats does not play a major role in the alterations of vascular tone observed during endotoxic shock.

PGI2 is opposed by Thromboxanes (“Tx”) and other prostaglandins that are associated with a prothrombotic state. Keeping the ratio of PGI2/Tx high is understood to be important in preventing a prothrombotic state, maintaining adequate vasodilation. In stressed states with adrenaline/epinephrine, platelet activation may accelerate prothrombin activity as well as driving thromboxane formation to the detriment of PGI2 formation.

Distal ischemia and necrosis have been reported with COVID-19 patients, as have elevated D-dimer fragment levels. D-dimer fragments are a hallmark of activated clotting, with thrombin degradation. Increased clotting is associated with bradykinin activation and, accordingly, indirectly affects vessel diameter.

COVID-19 patients are associated with worse outcomes if they are on ACE-I or ARB, and this is considered to be an issue with ACE2. ACE2 has been linked with COVID-19 virus attachment and entry into the cells. It has been hypothesized that disruption of the balance between ACE and ACE2 would result in abnormal blood pressure control. Accordingly, ACE2 might protect against increases in blood pressure and ACE2 deficiency might lead to hypertension. Indeed, studies have shown that patients with hypertension showed marked ACE up-regulation and ACE2 down-regulation in both heart and kidney. ARBs are associated with upregulation of ACE2, which has led to the concern about increased COVID-19 susceptibility in patients taking ARBs. That is, ARB use may increase ACE2, which is associated with greater entry of the virus into the person. The data has shown that comorbidity for COVID-19 is HTN, heart disease, pulmonary issues, obesity and age >45. Even if such high risk patients are not on an ARB, it is likely that they may be susceptible to the virus due to microvascular hypoperfusion and local hypoxia. This would be a concern in microvascular peripheral vascular disease. The COVID 19 virus replicates in a hypoxic environment due to hypoxia inducible factor (HIF) and expression of a Furan spike, dimerizing the virus and initiating replication.

In COVID-19 infection, the spike protein subunit, S1 is accelerated in the presence of thrombin, a key promotor of blood clotting. More recent connections with the S1 subunit of the spike are associated with increased clotting and decreased fibrin degradation or impairment of fibrinolysis. Accordingly, an increase of thrombin in COVID-19 conditions precipitates even more dense clots and platelet activation.

Studies in 2020 have shown that COVID-19 patients on lovenox/heparin had shorter hospital stays and lower morbidity/mortality than patients without the medication. It is known that these medications will accelerate eNOS to convert 1-arginine into nitric oxide, a potent viricide, as well as a vasodilator. This may have antagonized the hypoxia and hindered viral replication. In these at-risk patients, the GABA-a/AT1 construct may be utilized to improve PGI2 vasodilation, especially in the extremities, as the net PGI2 production is driven higher in these vessels.

The inventor has determined that lovenox/heparins blunt the effect of factor Xa and/or activate antithrombin to slow down the cascade of worsening blood flow conditions in COVID-19 patients. The inventor has also surmised that the provision of DOAC would limit or stop the clotting cascade by interfering with factor Xa and, accordingly, hence thrombin formation.

In endotoxic induced dogs, plasma renin activity (PRA) increased after decline in blood pressure, and Prostaglandin (PG) E-like material was observed in renal venous blood 30 minutes after endotoxin administration and was abolished by indomethacin. Endotoxin administration caused a substantial increase in the diameter of intrarenal arterial branches which was temporally associated with the appearance of PGE-like material in the renal venous effluent. The mesenteric arteries were initially and transiently constricted by endotoxin and then were markedly and chronically dilated. Indomethacin simultaneously abolished renal PG and decreased renal and mesenteric arterial diameter. This suggests the mesenteric and peripheral systemic vessels would benefit from increased PGI2 generation through the GABA-a/AT1 construct—especially when in septic state caused by COVID-19 or another infection.

The higher value circuits may still require the AT2-NO pathway for sufficiently protective vasodilation. Indeed, whereas both systemic and higher value circuits may utilize both PGI2 and EDRF for vasodilation, higher circuits typically rely more on EDRF and systemic circuits typically rely more on PGI2 lower. As noted above, in adult higher value circuits, increased PGI2 release may be induced by the provision of GABA-agonists when AII is bound to the AT1 receptor (mirroring the pregnant uterine vessel condition).

In hypotensive and normotensive septic states, AII may be high or normal. However, the administration of an ARB (and/or ACE-I), along with a GABA-a agonist, to promote increased PGI2 release in diseased adult systemic vessels may simultaneously reduce the release of PGI2 in the higher circuits. In such instances, the AT2-NO pathway may be rendered even more important to achieve sufficient vasodilation in the higher level circuits.

In an eHTN humans, there is low renin-low angiotensin II group, and low normal to normal renin-angiotensin II group and the high renin-angiotensin II group. COVID-19 patient may manifest the distal necrosis, if for example they are normal to high renin type eHTN. Such patients may possess the GABA-a/AT1 construct, and due to higher renin and angiotensin II, the peripheral circuit may be less dilated and/or more constricted. The higher AII will shut off the PGI2 pathway, but the onset of sepsis may cause transient dilation of the entire vasculature, driving more renin and AII. This, in turn, may cause peripheral vessels to become even smaller diameter as the higher circuits react to the higher AII to try to increase diameter. This ischemia may lead to distal necrosis.

In view of the above, the inventor has determined that the GABA-a/AT1 construct is at play in certain microvascular and sepsis-related disorders, and that exploitation of such mechanism may increase PGI2 release, and ultimately result in increased dilation of peripheral vessels. Such dilation may improve blood flow and, accordingly, treat, prevent, or reduce ischemia and/or other microvascular peripheral vascular disease and related conditions.

In some embodiments, a patient with COVID-19 or another condition associated with clotting cascade, may treated with a GABA-a agonist and ARB (and/or ACE-I) combination in addition to a DOAC in, for example, sub-therapeutic doses (with respect to medically accepted modes of use), to limit clotting issues while reducing blood pressure and/or improving blood flow.

### Metabolic Disorders

Prior to the work of the inventor, neither ACE-Is nor ARBs have been associated with increased PGI2. Indeed, in eHTN patients, the amount of AII is already so low that reducing it further via provision of ACE-Is should not have a significant contractile impact. Yet, reducing that remaining amount of AII is believed to reduce vessel contractility.

ACE-Is are believed to improve the fibrinolytic balance in eHTN patients by reducing plasma PAI-1 levels and, as a result, blood pressure. PAI-1 is an inhibitor of fibrin removal, a by-product of clotting. In the literature, ACE-Is have generally been considered to be more effective antihypertensives than ARBs in in diabetics. DM is characterized by issues with small vessel flow, clotting imbalances, and fibrin removal. Thus, the PAI-1 level-linked biological mechanisms relating to clotting and clot removal explains may explain the general conclusion in the literature is ACE-I produces better general outcomes in DM patients. Outside of DM, there are differences in pre-clotting states from ACE-I and ARBs and the literature has not been able to determine if one class is better than another.

The inventor considers DM to be, inter alia, a microangiopathic disease with alterations in vascular activity and permeability, increased clotting, and platelet activation. There are changes to basement membranes and barrier function at the endothelial level. The net effect is clotting activation and protein leakage through the small vessels. Maintenance of the capillary flow may permit more efficient delivery of sugars and insulin to the target peripheral tissues and thereby lower blood sugar levels.

DM and other metabolic disorders may be characterized by elevated circulating blood sugars, and chronic degenerative capillary disease in kidney, retina, myocardium/endocardium, and peripheral nerves. The inventor has surmised that DM may cause such vessels to mirror the GABA-a/AT1 construct of pregnant systemic vessels. Accordingly, the administration of ARB (and/or ACE-I) and a GABA-a agonist may induce increased PGI2 release in such vessels, and thereby improve the underlying condition via vasodilation that improves blood flow. It is contemplated that the additional administration of an AT2 agonist may support NO release via the AT2-NO pathway.

In normal conditions, adequate blood flow through peripheral and splanchnic/mesenteric vessels is maintained via both PGI2 and NO vasodilation, permitting normal system function.

EDRF formation has been shown to be a function of arginine metabolism, which is a keystone in metabolic syndrome. In certain metabolic syndromes, however, arginine metabolism from Arginine to nitric oxide may become disordered, resulting in non-vasodilator metabolites (e.g., symmetrical and asymmetrical dimethyl arginine) instead of EDRF. This may be referred to as spermine/spermidine metabolism.

Emerging evidence suggests that arginase is a key mediator of endothelial dysfunction in type 2 diabetes mellitus by reciprocally regulating nitric oxide bioavailability. Drivers of nitric oxide synthetase to make NO have been via the AT2 pathway or direct effect of heparins or low molecular weight heparin on NOS. NO synthetase promoters—including, but not limited to biopterin (BH4), arginine, and citrulline—may preferentially drive arginine metabolism towards NOS to make NO, a vasodilator. In other words, the NOS pathway may be accelerated by heparins/lovenox, but also by oral DOAC, biopterin (BH4), and other NO Synthetase promoters. This pathway converts l-arginine into nitric oxide through NOS. Similarly, Arginase inhibitors—including, but not limited Ornithine and Valine—may preferentially drive arginine metabolism away from away from spermine/spermidine metabolism—and thereby indirectly towards NO synthetase production. The effect of these NO synthetase promoters and/or arginase inhibitors can be utilized in concert with the GABA-a/AT1 construct.

In systemic vessels and lower level circuits, the blockade of the AT1 receptor may drive AII to the AT2 receptor, inducing NO release. Accordingly, exploitation of the GABAs/AT1 construct via an ARB and a GABA-a agonist may induce release of both PGI2 and NO—especially where an arginase inhibitor and/or NO synthetase promoters is provided to further support. In turn, this may support local microvascular flow, and relieve certain microvascular disorders.

In higher level circuits, the blockade of the AT1 receptor may drive AII to the AT2 receptor, inducing NO release. Accordingly, in higher level circuits, provision of an arginase inhibitor and/or NO synthetase promoter in conjunction with an ARB may sufficiently support EDRF release, even as the AT1 receptor blockage may hinder PGI2 release in these higher level circuits. An ACE-I may be used in this combination in addition to or instead of an ARB, but may less effective because the ACE-I may reduce AII to AT2 binding, and thereby drive down EDRF formation.

Regardless of whether an arginine metabolism disorder may be at play, co-administration of an arginase inhibitor and/or NO synthetase promoter along with an ARB (and/or ACE-i) and a GABA-a agonist may beneficially increase both EDRF and PGI2. Adding an AT2 agonist to this co-administration may further improve EDRF release.

For reasons similar to those discussed above regarding COVID-19, the inventor has determined that the provision of DOACs may improve capillary flow in DM patients.

In some embodiments, a patient with metabolic syndrome, such as adult onset diabetes, treated with a GABA-a agonist and ARB (and/or ACE-I) combination may further be provided with DOAC and/or an arginase inhibitor in, for example, sub-therapeutic doses, to improve blood sugar control and hemoglobin A1 C levels while reducing blood pressure and/or improving blood flow.

In other words, a composition may comprise (a) GABA-a agonist, (b) ARB (and/or ACE-I), and (c) an arginase inhibitor and/or NO synthetase promoter in a low dose amount. And such composition may be provided to identified diabetic (or, in some circumstances, pre-diabetic) patients. The addition of element (c) is believed to have a synergistic (and not merely additive) effect on diabetic patient because such compounds contribute to NOS activity and Nitric Oxide formation, which may improve blood flow to peripheral circuits, improve delivery of insulin and sugar to distal tissues for cellular glucose uptake, and thereby lower blood sugar and ultimately lower circulating insulin levels and fat metabolism.

Although the foregoing embodiments have been described in detail by way of illustration and example for purposes of clarity of understanding, it will be readily apparent to those of ordinary skill in the art in light of the description herein that certain changes and modifications may be made thereto without departing from the spirit or scope of the appended claims. It is also to be understood that the terminology used herein is for the purpose of describing particular aspects only, and is not intended to be limiting, since the scope of the present invention will be limited only by the appended claims.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of ordinary skill in the art upon reading this disclosure, each of the individual aspects described and illustrated herein has discrete components and features which may be readily separated from or combined with the features of any of the other several aspects without departing from the scope or spirit of the disclosure. Any recited method can be carried out in the order of events recited or in any other order that is logically possible. Accordingly, the preceding merely provides illustrative examples. It will be appreciated that those of ordinary skill in the art will be able to devise various arrangements which, although not explicitly described or shown herein, embody the principles of the disclosure and are included within its spirit and scope.

Furthermore, all examples and conditional language recited herein are principally intended to aid the reader in understanding the principles of the invention and the concepts contributed by the inventors to furthering the art, and are to be construed without limitation to such specifically recited examples and conditions. Moreover, all statements herein reciting principles and aspects of the invention, as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary configurations shown and described herein.

In this specification, various preferred embodiments have been described with reference to the accompanying drawings. It will be apparent, however, that various other modifications and changes may be made thereto and additional embodiments may be implemented without departing from the broader scope of the claims that follow. The specification and drawings are accordingly to be regarded in an illustrative rather than restrictive sense.

We claim:

1. A medical composition for treatment of a peripheral vascular disease, the medical composition comprising:
a first dosage of a GABA-a agonist;
a second dosage of an Angiotensin II modulator, the Angiotensin II modulator comprising at least one of an ARB and an ACE Inhibitor, the ARB being an Angiotensin II, type 1 receptor antagonist; and
a third dosage of a Direct Oral Anticoagulant.

2. The medical composition of claim 1, wherein:
the second dosage of the Angiotensin II modulator comprises the ARB.

3. The medical composition of claim 2, further comprising:
a fourth dosage of at least one NO synthetase promoter.

4. The medical composition of claim 3, wherein the at least one NO synthetase promoter comprises at least one of biopterin (BH4), arginine, and citrulline.

5. The medical composition of claim 2, further comprising:
a fourth dosage of at least one arginase inhibitor.

6. The medical composition of claim 3, wherein the at least one arginase inhibitor comprises at least one of Ornithine and Valine.

7. The medical composition of claim 1, further comprising:
a fourth dosage of at least one of a NO synthetase promoter and an arginase inhibitor; and
a fifth dosage of at least one AT2 agonist.

8. The medical composition of claim 1, further comprising:
a fourth dosage of at least one AT2 agonist.

9. The medical composition of claim 8, wherein:
the second dosage of the Angiotensin II modulator consists of at least one of Saralasin and Sarile; and
the fourth dosage comprises at least one of Saralasin and Sarile.

10. The medical composition of claim 8, wherein:
the fourth dosage comprises at least one of C21 peptides, Saralasin, and Sarile.

11. The medical composition of claim 1, wherein the third dosage of the Direct Oral Anticoagulant is of an amount that is sub-therapeutic with respect to other medically accepted modes of use.

12. The medical composition of claim 1, further comprising:
a fourth dosage of at least one EO-regulating compound.

13. A method for treating a patient with a microvascular disease condition, comprising:
providing the patient;
providing a medical composition, the medical composition including:
a first dosage of a GABA-a agonist;
a second dosage of an Angiotensin II modulator, the Angiotensin II modulator comprising at least one of an ARB and an ACE Inhibitor, the ARB being an Angiotensin II, type 1 receptor antagonist,
a third dosage of a Direct Oral Anticoagulant;
administering the medical composition to the patient.

14. The method of claim 13, further comprising:
reducing at least one of bradykinin levels and clotting agents through action of the third dose; and
increasing prostacyclin release in blood vessels affected by the vascular disease condition through combined action of the first and second dosages.

15. The method of claim 14, wherein, in the step of providing the medical composition, the medical composition includes a fourth dosage of at least one NO synthetase promoter, the method further comprising:
increasing EDRF release through action of the fourth dosage.

16. The method of claim 14, wherein, in the step of providing the medical composition, the medical composition includes a fourth dosage of at least one arginase inhibitor, the method further comprising:
increasing EDRF release through action of the fourth dosage.

17. The method of claim 14, wherein, in the step of providing the medical composition, the medical composition includes a fourth dosage of at least one AT2 agonist, the method further comprising:
increasing EDRF release through action of the fourth dosage.

18. The method of claim 13, wherein the step of providing the patient further comprises:
providing a patient with a microvascular disease condition of at least one of systemic sclerosis, vascular insufficiency, and distal ischemia.

19. The method of claim 18, wherein the step of providing the patient further comprises:
providing a patient infected with COVID-19.

20. The method of claim 18, wherein the step of providing the patient further comprises:
providing a patient with sepsis.
